(19) United States
(12) Patent Application Publication
(10) Pub. No.: US 2008/0269226 A1
(43) Pub. Date: Oct. 30, 2008

(54) Methods for Preserving Renal Function Using Xanthine Oxidoreductase Inhibitors

(76) Inventors: Christopher Lademacher, Chicago, IL (US); Patricia MacDonald, Skokie, IL (US)

Correspondence Address: Lisa V. Mueller;Dykema Gossett PLLC, Suite 2300, 10 South Wacker Drive, Chicago, IL, 60606, US

(21) Appl. No.: 11/939,112

(22) Filed: Nov. 13, 2007

Related U.S. Application Data
(60) Provisional application No. 60/858,509, filed on Nov. 13, 2006.

(51) Int. Cl. A61K 31/5365; A61K 31/381; A61P 13/12

(52) U.S. Cl. 514/246; 514/448

(57) ABSTRACT

The present invention relates to methods of preserving renal function in a subject in need thereof by administering a therapeutically effective amount of at least one xanthine oxidoreductase inhibiting compound or salt thereof.

### RELATED APPLICATION INFORMATION

This application claims the benefit of 60/858,509 filed on Nov. 13, 2006, the contents of which are herein incorporated by reference.

### FIELD OF THE INVENTION

The present invention relates to methods of treating subjects in order to preserve renal function. More specifically, the present invention involves administering to a subject in need of preservation of renal function a therapeutically effective amount of at least one xanthine oxidoreductase inhibiting compound or salt thereof in order to preserve the renal function of such patients.

### BACKGROUND OF THE INVENTION

It has been observed that subjects with conditions such as hyperuricemia, gout, acute gouty arthritis, chronic gouty joint disease, tophaceous gout, uric acid nephropathy, and/or nephrolithiasis (kidney stones) can sometimes suffer from a reduction of, or an impairment in, renal function, particularly as the conditions progress over time (See, Johnson, Blood Purif., 24:67-70 (2006), Siu, L., et al., AJKD, 47(1):51-99 (2006) and Iseki, I., et al., AJKD, 44(4):642-650 (2004)).

In general, subjects are viewed as having normal renal function when their serum creatinine levels are ≦1.5 mg/dL and their creatinine clearance is ≧50 mL/min. If the serum creatinine level becomes greater than 1.5 mg/dL, or if the creatinine clearance falls below 50 mL/min., the subject is deemed to be renally impaired. Another important measure of renal function is glomerular filtration rate or GFR. GFR is calculated by comparing urine creatinine levels with blood test results and is believed to give a more precise indication of the state of the kidneys. For most patients, a GFR over 60 ml/minute is adequate. If the GFR has significantly declined from a previous test result, however, this can be an early indicator of kidney disease requiring medical intervention.

In animal models, renal function can be assessed by measuring urinary protein excretion and glomerular hemodynamics (include whole kidney GFR, single-nephron GFR, glomerular pressure and flow, afferent resistance and efferent resistance) using renal micropuncture technique, among other methods known to those skilled in the art. In addition, renal histological evaluation for vacuolar degeneration of renal proximal tubules, tubulointerstitial fibrosis and thickening of the afferent arteriolar vascular wall can be used to further understand the causes or etiology of renal diseases.

Gout is characterized by the symptomatic deposition of urate crystals in joint tissues as a result of urate supersaturation of extracellular fluids, a biochemical aberration reflected by hyperuricemia (serum urate levels exceeding 7.0 mg/dL in men and exceeding 6.0 mg/dL in women). In patients with gout, renal calculi or “stones” occur with a frequency of 10-25% and in those patients approximately 1% will manifest the development of a uric acid renal calculus on an annual basis.

Long-term restoration of normal serum urate levels typically requires the use of an anti-hyperuricemic agent. Uric acid lowering therapy is recommended for subjects suffering from gout and one or more of the following conditions: acute gouty arthritis, chronic gouty joint disease, tophaceous gout, uric acid nephropathy, and/or nephrolithiasis (kidney stones). Although various therapies for reducing serum urate levels are known, their impact on renal function is not fully understood.

### SUMMARY OF THE PRESENT INVENTION

In one embodiment, the present invention relates to a method of preserving renal function in a subject in need thereof, the method including the step of administering to the subject a therapeutically effective amount of a xanthine oxidoreductase inhibitor or a pharmaceutically acceptable salt thereof.

In another embodiment, the present invention relates to a method of preserving renal function in a subject in need thereof, the method comprising the step of administering to the subject a therapeutically effective amount of a compound or a pharmaceutically acceptable salt thereof, wherein said compound comprises the formula:

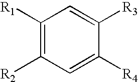

wherein R₁ and R₂ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, a phenylsulfinyl group or a cyano (—CN) group;

wherein R₃ and R₄ are each independently a hydrogen or A, B, C or D as shown below:

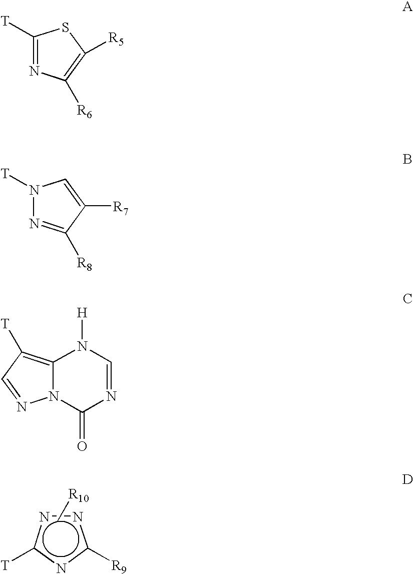

wherein T connects A, B, C or D to the aromatic ring shown above at R₁, R₂, R₃ or R₄.

wherein R₅ and R₆ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;

wherein R₇ and R₈ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;

wherein R₉ is an unsubstituted pyridyl group or a substituted pyridyl group; and

wherein R₁₀ is a hydrogen or a lower alkyl group, a lower alkyl group substituted with a pivaloyloxy group and in each case, R₁₀ bonds to one of the nitrogen atoms in the 1,2,4-triazole ring shown above.

In yet another embodiment, the present invention relates to a method of preserving renal function in a subject in need of thereof, the method comprising the step of administering to the subject a therapeutically effective amount of a compound or a pharmaceutically acceptable salt thereof, wherein said compound comprises the formula:

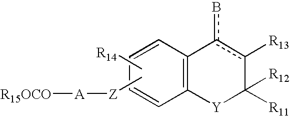

wherein R₁₁ and R₁₂ are each independently a hydrogen, a substituted or unsubstituted lower alkyl group, a substituted or unsubstituted phenyl, or R₁₁ and R₁₂ may together form a four- to eight-membered carbon ring together with the carbon atom to which they are attached;

wherein R₁₃ is a hydrogen or a substituted or unsubstituted lower alkyl group;

wherein R₁₄ is one or two radicals selected from a group consisting of a hydrogen, a halogen, a nitro group, a substituted or unsubstituted lower alkyl, a substituted or unsubstituted phenyl, —OR₁₆ and —SO₂NR₁₇R_{17′}, wherein R₁₆ is a hydrogen, a substituted or unsubstituted lower alkyl, a phenyl-substituted lower alkyl, a carboxymethyl or ester thereof, a hydroxyethyl or ether thereof, or an allyl; R₁₇ and R_{17′} are each independently a hydrogen or a substituted or unsubstituted lower alkyl;

wherein R₁₅ is a hydrogen or a pharmaceutically active ester-forming group;

wherein A is a straight or branched hydrocarbon radical having one to five carbon atoms;

wherein B is a halogen, an oxygen, or a ethylenedithio;

wherein Y is an oxygen, a sulfur, a nitrogen or a substituted nitrogen;

wherein Z is an oxygen, a nitrogen or a substituted nitrogen; and

the dotted line refers to either a single bond, a double bond, or two single bonds.

A subject being treated pursuant to the methods of the invention can have one or more of the following conditions: hyperuricemia, gout, acute gouty arthritis, chronic gouty joint disease, tophaceous gout, uric acid nephropathy, or nephrolithiasis. Alternatively, the subject may be suffering from a progressive renal disease, including, but not limited to, renal tubulointerstitial diseases, renal tubular cell injury, nephritis, glomerular diseases, glomerulonephritides, renal ischemia, renal ischemia/reperfusion injury, renal vascular diseases, renal artery or vein thrombosis, interstitial nephritis, toxic glomerulophathies, renal stones/nephrolithiasis, long standing hypertension, diabetic nephropathy, congestive heart failure, nephropathy from sickle cell anemia and other blood dyscrasias, nephropathy related to hepatitis, HIV, parvovirus and BK virus (a human polyomavirus), cystic kidney diseases, lupus nephritis, membranous glomerulonephritis, membranoproliferative glomerulonephritis, focal glomerular sclerosis, vasculitis, cryoglobulinemia, Anti-Neutrophil Cytoplasmic Antibody (ANCA)-positive vasculitis, ANCA-negative vasculitis, amyloidosis, multiple myeloma, renal light chain deposition disease, complications of kidney transplant, chronic rejection of a kidney transplant, chronic allograft nephropathy, and the chronic renal effects of immunosuppressives. Subjects being treated can also have impaired renal function as measured by known medical test methods. For example, subjects being treated can have a serum creatinine level of >1.5 mg/dL or a creatinine clearance of <50 mL/minute. Similarly, subjects being treated can have a GFR of <60 mg/minute. However, the subject being treated by the methods of the invention need not have any particular condition or impairment if it is determined that preservation or stabilization of renal function is medically necessary or desirable.

### BRIEF DESCRIPTION OF THE FIGURES

FIG. 1 shows the effect of febuxostat (Fx) on body weight (BW) in remnant kidney (RK) rats with and without coexisting oxonic acid (OA)-induced hyperuricemia.-- shows the BW of RK rats only (control); -∘- shows the BW of RK rats treated with Fx; -▪- shows the BW of RK rats treated with OA; and -□- shows the BW of RK treated with OA and Fx.

FIG. 2 shows the effect of febuxostat (Fx) on plasma uric acid (UA) in remnant kidney (RK) rats with and without coexisting oxonic acid (OA)-induced hyperuricemia.-- shows the UA of RK rats only (control); -∘- shows the UA of RK rats treated with Fx; -▪- shows the UA of RK rats treated with OA; and -□- shows the UA of RK treated with OA and Fx.

FIG. 3 shows the effect of febuxostat (Fx) on systolic blood pressure (SBP) in remnant kidney (RK) rats with and without coexisting oxonic acid (OA)-induced hyperuricemia. -- shows the SBP of RK rats only (control); -∘- shows the SBP of RK rats treated with Fx; -▪- shows the SBP of RK rats treated with OA; and -□- shows the SBP of RK treated with OA and Fx.

FIG. 4 shows the effect of febuxostat (Fx) on mean arterial pressure (under anesthesia) in remnant kidney (RK) rats with and without coexisting oxonic acid (OA)-induced hyperuricemia.

FIG. 5 shows the effect of febuxostat (Fx) on proteinuria in remnant kidney (RK) rats with and without coexisting oxonic acid (OA)-induced hyperuricemia. -- shows the proteinuria of RK rats only (control); -∘- shows the proteinuria of RK rats treated with Fx; -▪- shows the proteinuria of RK rats treated with OA; and -□- shows the proteinuria of RK treated with OA and Fx.

FIG. 6 shows the effect of febuxostat (Fx) on glomerular filtration rate in remnant kidney (RK) rats with and without coexisting oxonic acid (OA)-induced hyperuricemia.

FIG. 7 shows the effect of febuxostat (Fx) on glomerular hemodynamics in remnant kidney (RK) rats with and without coexisting oxonic acid (OA)-induced hyperuricemia.

FIG. 8 shows the effect of febuxostat (Fx) on renal arteriolar morphology in remnant kidney (RK) rats with and without coexisting oxonic acid (OA)-induced hyperuricemia.

FIG. 9 shows the effect of febuxostat (Fx) on renal tubulointerstitial fibrosis in remnant kidney (RK) rats with and without coexisting oxonic acid (OA)-induced hyperuricemia.

### DETAILED DESCRIPTION OF THE INVENTION

### Definitions

The terms “administer”, “administering”, “administered” or “administration” refer to any manner of providing a drug (such as, a xanthine oxidoreductase inhibitor or a salt thereof) to a subject or patient. Routes of administration can be accomplished through any means known by those skilled in the art. Such means include, but are not limited to, oral, buccal, intravenous, subcutaneous, intramuscular, by inhalation and the like.

As used herein, the phrases “progressive renal disease”, “end stage renal disease”, “chronic renal failure (CRF)”, “chronic renal disease (CRD)”, “chronic kidney disease (CKD)” which are all used interchangeably herein, refer to any kidney condition or dysfunction that occurs over a period of time, as opposed to a sudden event (namely, acute renal disease or renal failure), to cause a gradual decrease of renal function in a subject. For example, progressive renal disease, end stage renal disease, chronic kidney disease or chronic renal injury, includes, but is not limited to, conditions or dysfunctions caused by renal tubulointerstitial diseases, renal tubular cell injury, chronic infections, chronic inflammation, nephritis, glomerular diseases, glomerulonephritides, renal ischemia, renal ischemia/reperfusion injury, vascular diseases, renal artery or vein thrombosis, interstitial nephritis, drugs, toxins, trauma, renal stones/nephrolithiasis, chronic hyperuricemia, long standing hypertension, diabetes, congestive heart failure, nephropathy from sickle cell anemia and other blood dyscrasias, nephropathy related to hepatitis, HIV, parvovirus and BK virus (a human polyomavirus), cystic kidney diseases, congenital malformations, obstruction, malignancy, kidney disease of indeterminate causes, lupus nephritis, membranous glomerulonephritis, membranoproliferative glomerulonephritis, focal glomerular sclerosis, vasculitis, cryoglobulinemia, Anti-Neutrophil Cytoplasmic Antibody (ANCA)-positive vasculitis, ANCA-negative vasculitis, amyloidosis, multiple myeloma, light chain deposition disease, complications of kidney transplant, chronic rejection of a kidney transplant, chronic allograft nephropathy, and the chronic effects of immunosuppressives.

As used herein, the term “pharmaceutically acceptable” includes moieties or compounds that are, within the scope of sound medical judgment, suitable for use in contact with the tissues of humans and lower animals without undue toxicity, irritation, allergic response, and the like, and are commensurate with a reasonable benefit/risk ratio.

As used herein, the term “subject” refers to an animal, preferably a mammal, including a human or non-human. The terms patient and subject may be used interchangeably herein.

The terms “therapeutically effective amount” or “prophylactically effective amount” of a drug (namely, at least one xanthine oxidoreductase inhibitor or a salt thereof) refers to a nontoxic but sufficient amount of the drug to provide the desired effect of preserving renal function in a subject. In other words, these terms mean a sufficient amount of, for example, the composition, xanthine oxidoreductase inhibiting compound, or formulation necessary to preserve the subject's renal function, at a reasonable benefit/risk ratio applicable to any medical treatment. As with other pharmaceuticals, it will be understood that the total daily usage of a pharmaceutical composition of the invention will be decided by a patient's attending physician within the scope of sound medical judgment. The specific therapeutically effective or prophylactically effective dose level for any particular patient will depend upon a variety of factors including the disorder being treated and the severity of the disorder; activity of the specific compound employed; the specific composition employed; the age, body weight, general health, sex and diet of the patient; the time administration, route of administration, and rate of excretion of the specific compound employed; the duration of the treatment; drugs used in combination or coincidental with the specific compound employed; and other factors known to those of ordinary skill in the medical arts. For example, it is well within the skill of the art to start doses of the compound at levels lower than required to achieve the desired therapeutic effect and to gradually increase the dosage until the desired effect is achieved.

Accordingly, the amount of drug that is “effective” or “prophylactic” will vary from subject to subject, depending on the age and general condition of the individual, the particular drug or drugs, and the like. Thus, it is not always possible to specify an exact “therapeutically effective amount” or a “prophylactically effective amount”. However, an appropriate “therapeutically effective amount” or “prophylactically effective amount” in any individual case may be determined by one skilled in the art.

The terms “treating” and “treatment” refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual by inhibiting or causing regression of a disorder or disease.

As used herein, the term “xanthine oxidoreductase inhibitor” refers to any compound that (1) is an inhibitor of a xanthine oxidoreductase, such as, but not limited to, xanthine oxidase; and (2) chemically, does not contain a purine ring in its structure (i.e. is a “non-purine”). The phrase “xanthine oxidoreductase inhibitor” as defined herein also includes metabolites, polymorphs, solvates and prodrugs of the such compounds, including metabolites, polymorphs, solvates and prodrugs of the exemplary compounds described as Formula I and Formula II below. Examples of xanthine oxidoreductase inhibitors include, but are not limited to, 2-[4-(2-carboxypropoxy)-3-cyanophenyl]-4-methyl-5-thiazolecarboxylic acid and compounds having the following Formula I or Formula II:

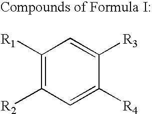

wherein R₁ and R₂ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, a phenylsulfinyl group or a cyano (—CN) group;

wherein R₃ and R₄ are each independently a hydrogen or A, B, C or D as shown below:

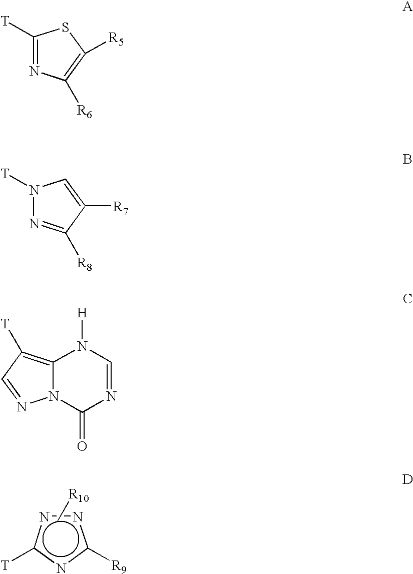

wherein T connects or attaches A, B, C or D to the aromatic ring shown above at R₁, R₂, R₃ or R₄.

wherein R₅ and R₆ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;

wherein R₇ and R₈ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;

wherein R₉ is an unsubstituted pyridyl group or a substituted pyridyl group; and

wherein R₁₀ is a hydrogen or a lower alkyl group, a lower alkyl group substituted with a pivaloyloxy group and in each case, R₁₀ bonds to one of the nitrogen atoms in the 1,2,4-triazole ring shown above in Formula I.

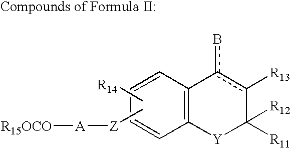

wherein R₁₁ and R₁₂ are each independently a hydrogen, a substituted or unsubstituted lower alkyl group, a substituted or unsubstituted phenyl (the substituted phenyl in this Formula II refers to a phenyl substituted with a halogen or lower alkyl, and the like. Examples include, but are not limited to, p-tolyl and p-chlorophenyl), or R₁₁ and R₁₂ may together form a four- to eight-membered carbon ring together with the carbon atom to which they are attached;

wherein R₁₃ is a hydrogen or a substituted or unsubstituted lower alkyl group;

wherein R₁₄ is one or two radicals selected from a group consisting of a hydrogen, a halogen, a nitro group, a substituted or unsubstituted lower alkyl group, a substituted or unsubstituted phenyl (the substituted phenyl in this Formula II refers to a phenyl substituted with a halogen or lower alkyl group, and the like. Examples include, but are not limited to, p-tolyl and p-chlorophenyl), —OR₁₆ and —SO₂NR₁₇R_{17′}, wherein R₁₆ is a hydrogen, a substituted or unsubstituted lower alkyl, a phenyl-substituted lower alkyl, a carboxymethyl or ester thereof, a hydroxyethyl or ether thereof, or an allyl; R₁₇ and R_{17′} are each independently a hydrogen or a substituted or unsubstituted lower alkyl group;

wherein R₁₅ is a hydrogen or a pharmaceutically active ester-forming group;

wherein A is a straight or branched hydrocarbon radical having one to five carbon atoms;

wherein B is a halogen, an oxygen, or a ethylenedithio;

wherein Y is an oxygen, a sulfur, a nitrogen or a substituted nitrogen;

wherein Z is an oxygen, a nitrogen or a substituted nitrogen; and

the dotted line refers to either a single bond, a double bond, or two single bonds (for example, when B is ethylenedithio, the dotted line shown in the ring structure can be two single bonds).

As used herein, the term “lower alkyl(s)” group refers to a C₁-C₇ alkyl group, including, but not limited to, including methyl, ethyl, n-propyl, isopropyl, butyl, isobutyl, sec-butyl, tert-butyl, pentyl, isopentyl, hexyl, heptal and the like.

As used herein, the term “lower alkoxy” refers to those groups formed by the bonding of a lower alkyl group to an oxygen atom, including, but not limited to, methoxy, ethoxy, propoxy, isopropoxy, butoxy, isobutoxy, pentoxy, hexoxy, heptoxy and the like.

As used herein, the term “lower alkylthio group” refers to those groups formed by the bonding of a lower alkyl to a sulfur atom.

As used herein, the term “halogen” refers to fluorine, chlorine, bromine and iodine.

As used herein, the term “substituted pyridyl” refers to a pyridyl group that can be substituted with a halogen, a cyano group, a lower alkyl, a lower alkoxy or a lower alkylthio group.

As used herein, the term “four- to eight-membered carbon ring” refers to cyclobutyl, cyclopentyl, cyclohexyl, cycloheptyl, cyclooctyl and the like.

As used herein, the phrase “pharmaceutically active ester-forming group” refers to a group which binds to a carboxyl group through an ester bond. Such ester-forming groups can be selected from carboxy-protecting groups commonly used for the preparation of pharmaceutically active substances, especially prodrugs. For the purpose of the invention, said group should be selected from those capable of binding to compounds having Formula II wherein R₁₅ is hydrogen through an ester bond. Resultant esters are effective to increase the stability, solubility, and absorption in gastrointestinal tract of the corresponding non-esterified forms of said compounds having Formula II, and also prolong the effective blood-level of it. Additionally, the ester bond can be cleaved easily at the pH of body fluid or by enzymatic actions in vivo to provide a biologically active form of the compound having Formula II. Preferred pharmaceutically active ester-forming groups include, but are not limited to, 1-(oxygen substituted)-C₂ to C₁₅ alkyl groups, for example, a straight, branched, ringed, or partially ringed alkanoyloxyalkyl groups, such as acetoxymethyl, acetoxyethyl, propionyloxymethyl, pivaloyloxymethyl, pivaloyloxyethyl, cyclohexaneacetoxyethyl, cyclohexanecarbonyloxycyclohexylmethyl, and the like, C₃ to C₁₅ alkoxycarbonyloxyalkyl groups, such as ethoxycarbonyloxyethyl, isopropoxycarbonyloxyethyl, isopropoxycarbonyloxypropyl, t-butoxycarbonyloxyethyl, isopentyloxycarbonyloxypropyl, cyclohexyloxycarbonyloxyethyl, cyclohexylmethoxycarbonyloxyethyl, bornyloxycarbonyloxyisopropyl, and the like, C₂ to C₈ alkoxyalkyls, such as methoxy methyl, methoxy ethyl, and the like, C₄ to C₈ 2-oxacycloalkyls such as, tetrahydropyranyl, tetrahydrofuranyl, and the like, substituted C₈ to C₁₂ aralkyls, for example, phenacyl, phthalidyl, and the like, C₆ to C₁₂ aryl, for example, phenyl xylyl, indanyl, and the like, C₂ to C₁₂ alkenyl, for example, allyl, (2-oxo-1,3-dioxolyl)methyl, and the like, and [4,5-dihydro-4-oxo-1H-pyrazolo[3,4-d]pyrimidin-1-yl]methyl, and the like.

In R₁₆ in Formula II, the term “ester” as used in the phrase “the ester of carboxymethyl” refers to a lower alkyl ester, such as methyl or ethyl ester; and the term “ether” used in the phrase “the ether of hydroxyethyl” means an ether which is formed by substitution of the hydrogen atom of hydroxyl group in the hydroxyethyl group by aliphatic or aromatic alkyl group, such as benzyl.

The carboxy-protecting groups may be substituted in various ways. Examples of substituents include halogen atom, alkyl groups, alkoxy groups, alkylthio groups and carboxy groups.

As used herein, the term “straight or branched hydrocarbon radical” in the definition of A in Formula II above refers to methylene, ethylene, propylene, methylmethylene, or isopropylene.

As used herein, the substituent of the “substituted nitrogen” in the definition of Y and Z in Formula II above are hydrogen, lower alkyl, or acyl.

As used herein, the term “phenyl-substituted lower alkyl” refers to a lower alkyl group substituted with phenyl, such as benzyl, phenethyl or phenylpropyl.

As used herein, the term “prodrug” refers to a derivative of the compounds shown in the above-described Formula I and Formula II that have chemically or metabolically cleavable groups and become by solvolysis or under physiological conditions compounds that are pharmaceutically active in vivo. Esters of carboxylic acids are an example of prodrugs that can be used in the dosage forms of the present invention. Methyl ester prodrugs may be prepared by reaction of a compound having the above-described formula in a medium such as methanol with an acid or base esterification catalyst (e.g., NaOH, H₂SO₄). Ethyl ester prodrugs are prepared in similar fashion using ethanol in place of methanol.

Examples of compounds having the above Formula I are: 2-[3-cyano-4-(2-methylpropoxy)phenyl]-4-methylthiazole-5-carboxylic acid (also known as “febuxostat”), 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid, 1-(3-cyano-4-(2,2-dimethylpropoxy)phenyl)-1H-pyrazole-4-carboxylic acid, 1-3-Cyano-4-(2,2-dimethylpropoxy)phenyl]-1H-pyrazole-4-carboxylic acid, pyrazolo[1,5-a]-1,3,5-triazin-4-(1H)-one, 8-[3-methoxy-4-(phenylsulfinyl)phenyl]-sodium salt (±) or 3-(2-methyl-4-pyridyl)-5-cyano-4-isobutoxyphenyl)-1,2,4-triazole.

Preferred compounds having the above Formula I are: 2-[3-cyano-4-(2-methylpropoxy)phenyl]-4-methylthiazole-5-carboxylic acid, 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid. These preferred compounds have also been found not have an effect at a therapeutically effective amount in a subject on the activity of any of the following enzymes involved in purine and pyrimidine metabolism: guanine deaminase, hypoxanthine-guanine phosphoribosyltransferse, purine nucleotide phosphorylase, orotate phosphoribosyltransferase or orotidine-5-monophosphate decarboxylase (i.e., meaning that it is “selective” for none of these enzymes which are involved in purine and pyrimidine metabolism). Assays for determining the activity for each of the above-described enzymes is described in Yasuhiro Takano, et al., Life Sciences, 76:1835-1847 (2005). These preferred compounds have also been referred to in the literature as nonpurine, selective inhibitors of xathine oxidase (NP/SIXO).

Examples of compounds having the above Formula II are described in U.S. Pat. No. 5,268,386 and EP 0 415 566 A1.

With the exception of pyrazolo[1,5-a]-1,3,5-triazin-4-(1H)-one, 8-[3-methoxy-4-(phenylsulfinyl)phenyl]-sodium salt (±), methods for making xanthine oxidoreductase inhibiting compounds of Formulas I and II for use in the methods of the present invention are known in the art and are described, for example, in U.S. Pat. Nos. 5,268,386, 5,614,520, 6,225,474, 7,074,816 and EP 0 415 566 A1 and in the publications Ishibuchi, S. et al., Bioorg. Med. Chem. Lett., 11:879-882 (2001) and which are each herein incorporated by reference. Other xanthine oxidoreductase inhibiting compounds can be found using xanthine oxidoreductase and xanthine in assays to determine if such candidate compounds inhibit conversion of xanthine into uric acid. Such assays are well known in the art.

Pyrazolo[1,5-a]-1,3,5-triazin-4-(1H)-one, 8-[3-methoxy-4-(phenylsulfinyl)phenyl]-sodium salt (±) is available from Otsuka Pharmaceutical Co. Ltd. (Tokyo, Japan) and is described in the following publications: Uematsu T., et al., “Pharmacokinetic and Pharmacodynamic Properties of a Novel Xanthine Oxidase Inhibitor, BOF-4272, in Healthy Volunteers, J. Pharmacology and Experimental Therapeutics, 270:453-459 (August 1994), Sato, S., A Novel Xanthine Deydrogenase Inhibitor (BOF-4272). In Purine and Pyrimidine Metabolism in Man, Vol. VII, Part A, ed. By P. A. Harkness, pp. 135-138, Plenum Press, New York. Pyrazolo[1,5-a]-1,3,5-triazin-4-(1H)-one, 8-[3-methoxy-4-(phenylsulfinyl)phenyl]-sodium salt (±) can be made using routine techniques known in the art.

### DESCRIPTION OF THE INVENTION

As mentioned briefly above, the present invention relates to methods of preserving renal function in subjects in need thereof. It has been discovered that a class of compounds known as xanthine oxidoreductase inhibitors can be used not only to reduce serum urate levels in subjects, but also to preserve renal function in said subjects over time.

Because the xanthine oxidoreductase inhibitors of the present invention are effective in reducing serum urate levels, these compounds can be used to treat subjects suffering from hyperuricemia, gout, acute gouty arthritis, chronic gouty disease, tophaceous gout, uric acid nephropathy, and/or nephrolithiasis. Such treatments involve the administration of sufficient amounts of xanthine oxidoreductase inhibitor to reduce uric acid levels in the subject with a quick onset (namely, within one week of first beginning treatment with a xanthine oxidoreductase inhibitor (See, Becker M, Kisicki J, Khosravan R, Wu J, Mulford D, Hunt B, MacDonald P, Joseph-Ridge N., Nucleosides Nucleotides Nucleic Acids, 23(8 & 9): 1111-1116 (October 2004)) and maintain a reduction in the subject's serum urate level for a prolonged period, preferably for at least 4 weeks (See, Becker M A, Schumacher H R Jr, Wortmann R L, MacDonald P A, Palo W A, Eustace D, Vernillet L, Joseph-Ridge N, Arthritis Rheum., 52(3):916-923 (March 2005)), more preferably for at least a year, still more preferably for at least two years, and still more preferably for in excess of 30 months and beyond (See, Becker M A, Schumacher H R Jr, Wortmann R L, MacDonald P A, Eustace D, Palo W A, Streit J, Joseph-Ridge N., N Engl J Med., 354(6):1532-1533 (April 2006)).

It was discovered that administering xanthine oxidoreductase inhibitors in quantities that are effective to reduce a subject's serum urate level for such prolonged periods is also therapeutically effective in preserving the subject's renal function during such periods. Preservation of renal function can be assessed by well-known measures, such as creatinine levels, creatinine clearance, and the GFR. It will be understood that preservation of renal function entails not only better renal function in xanthine oxidoreductase inhibitor-treated subjects than in placebo-treated subjects, but also maintaining renal function reasonably close to baseline levels, i.e., at stable levels, not necessarily improving renal function from reduced or impaired levels to adequate levels. In other words, while administration of xanthine oxidoreductase inhibitors is effective to preserve renal function at the subject's existing levels, i.e., stabilize renal function, it is not necessarily effective to improve renal function significantly beyond those levels. Nevertheless, maintaining existing levels of renal function is of importance to subjects suffering from conditions like hyperuricemia, gout, acute gouty arthritis, chronic gouty disease, tophaceous gout, uric acid nephropathy, and/or nephrolithiasis, since it may slow the progression of kidney disease in such patients.

When GFR is used as the measure of renal function, preserving the subject's renal function involves maintaining the subject's GFR at a level of at least approximately 75% or greater when compared to the subject's baseline levels; more preferably, at a level of at least approximately 80% or greater when compared to the subject's baseline levels; and, still more preferably, at a level of at least approximately 90% or greater when compared to the subject's baseline levels.

In addition, it has also been found that the administration of the xanthine oxidoreductase inhibitors of the present invention can also be used to preserve the renal function in subjects suffering from progressive renal disease. Such subjects may or may not also be suffering from hyperuricemia, gout, acute gouty arthritis, chronic gouty disease, tophaceous gout, uric acid nephropathy, and/or nephrolithiasis. The treatment of subjects suffering from progressive renal disease involves the administration of sufficient amounts of xanthine oxidoreductase inhibitor to maintain or improve renal function in a subject with a quick onset (namely, within two weeks of first beginning treatment with a xanthine oxidoreductase inhibitor) and maintain such improved renal function in the subject for a prolonged period, preferably for at least 4 weeks, more preferably for at least a year, still more preferably for at least two years, and still more preferably for in excess of 30 months and beyond. The methods described previously herein for measuring the preservation of renal function can also be used to measure the preservation of renal function in subjects suffering from progressive renal disease. It will be understood that preservation of renal function entails not only better renal function in xanthine oxidoreductase inhibitor-treated subjects than in placebo-treated subjects, but also maintaining renal function reasonably close to baseline levels, i.e., at stable levels, not necessarily improving renal function from reduced or impaired levels to adequate levels. In other words, while administration of xanthine oxidoreductase inhibitors is effective to preserve renal function at the subject's existing levels, i.e., stabilize renal function, it is not necessarily effective to improve renal function significantly beyond those levels. Nevertheless, maintaining existing levels of renal function is of importance to subjects suffering from progressive renal disease, since it may slow the progression of the disease in such patients.

Compositions containing at least one xanthine oxidoreductase inhibitor are contemplated for use in the methods of the present invention. Using the excipients and dosage forms described below, formulations containing such combinations are a matter of choice for those skilled in the art. Further, those skilled in the art will recognize that various coatings or other separation techniques may be used in cases where the combination of compounds are incompatible.

Compounds for use in accordance with the methods of the present invention can be provided in the form of pharmaceutically acceptable salts derived from inorganic or organic acids. Pharmaceutically acceptable salts are well-known in the art. For example, S. M. Berge et al. describe pharmaceutically acceptable salts in detail in J. Pharmaceutical Sciences, 66: 1 et seq. (1977). The salts can be prepared in situ during the final isolation and purification of the compounds or separately by reacting a free base function with a suitable organic acid. Representative acid addition salts include, but are not limited to, acetate, adipate, alginate, citrate, aspartate, benzoate, benzenesulfonate, bisulfate, butyrate, camphorate, camphor sulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methane sulfonate, nicotinate, 2-naphthalene sulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

Basic addition salts can be prepared in situ during the final isolation and purification of compounds by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylammonium, dimethylammonium, trimethylammonium, triethylammonium, diethylammonium, and ethylammonium among others. Other representative organic amines useful for the formation of base addition salts include ethylenediamine, ethanolamine, diethanolamine, piperidine, piperazine and the like.

The at least one xanthine oxidoreductase inhibiting compound or salts thereof, may be formulated in a variety of ways that is largely a matter of choice depending upon the delivery route desired. For example, solid dosage forms for oral administration include capsules, tablets, pills, powders and granules. In such solid dosage forms, the xanthine oxidoreductase inhibiting compound may be mixed with at least one inert, pharmaceutically acceptable excipient or carrier, such as sodium citrate or dicalcium phosphate and/or a) fillers or extenders, such as, but not limited to, starches, lactose, sucrose, glucose, mannitol and silicic acid; b) binders, such as, but not limited to, carboxymethylcellulose, alginates, gelatin, polyvinylpyrrolidone, sucrose and acacia; c) humectants, such as, but not limited to glycerol; d) disintegrating agents, such as, but not limited to, agar-agar, calcium carbonate, potato or tapioca starch, alginic acid, certain silicates and sodium carbonate; e) solution retarding agents, such as, but not limited to, paraffin; f) absorption accelerators, such as, but not limited to, quaternary ammonium compounds; g) wetting agents, such as, but not limited to, cetyl alcohol and glycerol monostearate; h) absorbents, such as, but not limited to, kaolin and bentonite clay; and i) lubricants, such as, but not limited to, talc, calcium stearate, magnesium stearate, solid polyethylene glycols, sodium lauryl sulfate and mixtures thereof.

Solid compositions of a similar type may also be employed as fillers in soft and hard-filled gelatin capsules using such excipients as lactose or milk sugar as well as high molecular weight polyethylene glycols and the like.

The solid dosage forms of tablets, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

Liquid dosage forms for oral administration include pharmaceutically acceptable emulsions, solutions, suspensions, syrups and elixirs. In addition to the xanthine oxidoreductase inhibiting compounds, the liquid dosage forms may contain inert diluents commonly used in the art such as, for example, water or other solvents, solubilizing agents and emulsifiers, such as, but not limited to, ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

The compositions can also be delivered through a catheter for local delivery at a target site, via an intracoronary stent (a tubular device composed of a fine wire mesh), or via a biodegradable polymer.

Compositions suitable for parenteral injection may comprise physiologically acceptable, sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include, but are not limited to, water, ethanol, polyols (propylene glycol, polyethylene glycol, glycerol, and the like), vegetable oils (such as olive oil), injectable organic esters such as ethyl oleate, and suitable mixtures thereof.

These compositions can also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example, sugars, sodium chloride and the like. Prolonged absorption of the injectable pharmaceutical form can be brought about by the use of agents delaying absorption, for example, aluminum monostearate and gelatin.

Suspensions, in addition to the active compounds (i.e., xanthine oxidoreductase inhibiting compounds or salts thereof), may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Proper fluidity can be maintained, for example, by the use of coating materials such as lecithin, by the maintenance of the required particle size in the case of dispersions and by the use of surfactants.

In some cases, in order to prolong the effect of the drug (i.e. xanthine oxidoreductase inhibiting compounds or salts thereof), it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Dosage forms for topical administration of the compounds of this present invention include powders, sprays, ointments and inhalants. The active compound(s) is mixed under sterile conditions with a pharmaceutically acceptable carrier and any needed preservatives, buffers or propellants which can be required. Ophthalmic formulations, eye ointments, powders and solutions are also contemplated as being within the scope of this invention.

It will be understood that formulations used in accordance with the present invention generally will comprise a therapeutically effective amount of one or more xanthine oxidoreductase inhibiting compounds.

Formulations of the present invention are administered and dosed in accordance with sound medical practice, taking into account the clinical condition of the individual patient, the site and method of administration, scheduling of administration, and other factors known to medical practitioners.

Therapeutically effective or prophylactically effective amounts for purposes herein thus can readily be determined by such considerations as are known to those skilled in the art. The daily therapeutically effective or prophylactically effective amount of the xanthine oxidoreductase inhibiting compounds administered to a patient in single or divided doses range from about 0.01 to about 750 milligram per kilogram of body weight per day (mg/kg/day). More specifically, a patient may be administered from about 5.0 mg to about 300 mg once daily, preferably from about 20 mg to about 240 mg once daily and most preferably from about 40 mg to about 120 mg once daily of xanthine oxidoreductase inhibiting compounds. Of course, it will be understood by one skilled in the art that other dosage regimens may be utilized, such as dosing more than once per day, utilizing extended, controlled, or modified release dosage forms, and the like in order to achieve the desired result of preserving a subject's renal function.

By way of example, and not of limitation, examples of the present invention will now be given.

### EXAMPLE 1

Information was collected prospectively in a subgroup of 18 human subjects with a history of nephrolithiasis, as reported by the subjects prior to study enrollment. In a 4-week, double-blind, phase 2 study, subjects were randomly assigned to one or four treatment arms: (1) febuxostat 40 mg per day, (2) febuxostat 80 mg per day, (3) febuxostat 120 mg per day, or (4) placebo.

Subjects completing the double-blind study entered an open-label, long-term study and began treatment with 80 mg febuxostat per day. Febuxostat doses could be titrated over the initial 6 months to 40 mg or 120 mg febuxostat per day based on the subjects' serum urate levels and the occurrence of adverse events.

In the study subset, a post-hoc analysis of nephrolithiasis outcome in the study subjects (n=13) who had received febuxostat for ≧30 months. In the event of an occurrence of renal calculus formation, all such stones were analyzed for mineral content.

The following were the criteria for inclusion in the study: (1) a history or presence of gout as defined by the American Rheumatism Association Preliminary criteria; (2) normal renal function, defined as serum creatinine level ≦1.5 mg/dL and creatinine clearance of ≧50 mL/min.; (3) serum urate level of ≧8.0 mg/dL at the start of the double-blind study.

The following were the criteria for exclusion from the study: (1) history of active liver disease, xanthinuria, or any other significant medical condition; and (2) subjects who had any change in thiazide diuretic or steroid therapy within one month of study enrollment and chronic use of NSAIDs.

Table 1 provides a summary of the baseline characteristics for the 18 subjects observed.

TABLE 1

| | |
|---|---|
| | All Subjects |
| Baseline Characteristics | N = 18 |

Gender

| | |
|---|---|
| Male | 16 |
| Female | 2 |

Race

| | |
|---|---|
| White | 17 |
| Other | 1 |

Age (years)

| | |
|---|---|
| Mean (SD) | 55.1 (13.25) |
| Range | 32-80 |

BMI (kg/m²)

| | |
|---|---|
| Mean (SD) | 35.8 (6.44) |
| Range | 23-48 |

Co-Morbidity History^{a}

| | |
|---|---|
| Hypertension | 8 |
| Coronary Artery Disease | 2 |
| Hyperlipidemia | 6 |
| Obesity | 5 |

Gout History (years)

| | |
|---|---|
| 1-5 | 2 |
| 5-10 | 5 |
| >10 | 11 |

Alcohol Use

| | |
|---|---|
| Drinker (1 to 14 drinks/week) | 6 |

Previous Drug History for Treatment of Gout

| | |
|---|---|
| Allopurinol (50 mg qd-300 mg bid) | 9 |

Table 2 provides a summary of renal function measures and longer-term serum urate response in subjects completing >30 months of treatment.

TABLE 2

| | | | | | | Measured | | | | | | | | | | | | | | |
|---|---|---|---|---|---|---|---|---|---|---|---|---|---|---|---|---|---|---|---|---|
| | | | | Urine Uric | | Creatinine | | | | | | | | | | | | | | |
| | | Febuxostat | | Acid | | Clearance | | Serum Creatinine | | | | | Estimated GFR | | | | Serum Urate | | | |
| | | Dose (mg/day) | | (mg/day) | | (mL/minute) | | (mg/dL) | | | | | (mL/min) | | | | (mg/dL) | | | |
| | Calculus | DB | OL | DB | | DB | | DB | | OL^{c} | | | | OL^{c} | | | | OL^{c} | | |
| | History^{b} | Dose | Dose | | Wk | | Wk | | Wk | Yr | Yr | Yr | DB | Yr | Yr | Yr | DB | Yr | Yr | Yr |
| Subject#^{a} | (years) | (mg) | (mg) | BL | 4 | BL | 4 | BL | 4 | 1 | 2 | 3 | BL | 1 | 2 | 3 | BL | 1 | 2 | 3 |

Overproducers (Urine Uric Acid > 800 mg/day at BL)

| | | | | | | | | | | | | | | | | | | | | |
|---|---|---|---|---|---|---|---|---|---|---|---|---|---|---|---|---|---|---|---|---|
| 1 | 7.20 | 80 | 80 | 925 | 387 | 77 | 103 | 1.2 | 1.1 | 1.1 | 1.2 | 1.2 | 73 | 80 | 72 | 72 | 8.7 | 4.3 | 4.1 | 5.8 |
| 2^{d} | 14.30 | 80 | 80 | 975 | 504 | 101 | 147 | 1.3 | 1.2 | 1.2 | 1.1 | 1.2 | 67 | 73 | 80 | 72 | 9.7 | 5.9 | 4.2 | 3.2 |
| 3 | 11.39 | 80 | 80 | 941 | 319 | 97 | 93 | 1.2 | 1.1 | 1.2 | 1.1 | 1.2 | 67 | 67 | 74 | 66 | 8.7 | 4.4 | 4.8 | 3.9 |

Underexcretors (Urine Uric Acid < 800 mg/day at BL)

| | | | | | | | | | | | | | | | | | | | | |
|---|---|---|---|---|---|---|---|---|---|---|---|---|---|---|---|---|---|---|---|---|
| 4 | 3.67 | PL | 80 | 706 | 572 | 77 | 95 | 1.3 | 1.3 | 1.2 | 1.3 | 1.3 | 62 | 68 | 61 | 61 | 11.6 | 6.1 | 7.6 | 5.8 |
| 5 | 2.38 | 80 | 80 | 740 | 286 | 80 | 88 | 1.3 | 1.2 | 1.4 | 1.3 | 1.4 | 62 | 57 | 62 | 57 | 11.0 | 4.5 | 6.4 | 3.3 |
| 6 | 0.22 | 40 | 80 | 790 | 504 | 67 | 82 | 1.2 | 1.2 | 1.3 | 1.4 | 1.5 | 67 | 61 | 56 | 51 | 8.7 | 6.4 | 9.7 | 7.5 |
| 7 | 43.23 | PL | 80 | 420 | 487 | 55 | 57 | 1.7 | 1.6 | 1.4 | 1.3 | 1.4 | 43 | 54 | 59 | 54 | 11.2 | 4.1 | 5.4 | 4.6 |
| 8 | 28.25 | 120 | 80 | 202 | 17 | 43 | 38 | 1.2 | 1.4 | 1.6 | 1.4 | 1.4 | 47 | 33 | 39 | 39 | 9.2 | 3.6 | 4.7 | 3.8 |
| 9 | 40.23 | 80 | 80 | 420 | 235 | 58 | 63 | 1.7 | 1.5 | 1.5 | 1.5 | 1.8 | 43 | 49 | 49 | 40 | 11.6 | 7.4 | 6.0 | 6.8 |
| 10 | 8.44 | 80 | 80 | 420 | 185 | 54 | 64 | 1.2 | 1.1 | 1.1 | 1.1 | 1.2 | 66 | 73 | 73 | 65 | 10.7 | 4.6 | 4.4 | 3.8 |
| 11 | 0.41 | 80 | 80 | 286 | 403 | 110 | 94 | 1.0 | 1.2 | 1.2 | 1.1 | 1.2 | 84 | 68 | 75 | 67 | 9.2 | 4.4 | 5.7 | 5.2 |
| 12 | 40.95 | 40 | 120 | 504 | 202 | 61 | 56 | 1.2 | 1.3 | 1.2 | 1.3 | 1.3 | 63 | 63 | 58 | 57 | 8.3 | 3.1 | 3.0 | 3.3 |
| 13^{e} | 16.46 | PL | 120 | 555 | 925 | 59 | 62 | 1.2 | 1.2 | 1.0 | 1.1 | 1.5 | 68 | 84 | 75 | 52 | 13.4 | 4.6 | 4.2 | 3.8 |

DB = double-blind, OL = open-label, BL = baseline, Wk = week, Yr = year, PL = placebo, NA = not applicable
^{a}Of the 18 subjects in the subgroup, 5 subjects taking febuxostat prematurely discontinued the study with <6 months treatment.
^{b}Time from last pre-study kidney stone to first dose of febuxostat.
^{c}Cumulative study days were used for Year 1, Year 2; and Year 3; the closest values to Day 365 + ≦14 days (Year 1), to Day 730 + ≦14 days (Year 2), and to Day 995 (Year 3) were recorded.
^{d}Subject #2 had a calcium oxylate stone on Day 1005 of study with a sUA 4.2 mg/dL while receiving febuxostat 80 mg/day. This subject had a second calcium oxylate stone on Day 1265.
^{e}Subject #13 had a calcium oxylate stone on Day 17 of DB study with a sUA of 13.4 mg/dL while receiving placebo and an additional calcium oxylate stone on Day 38 of the OL study while receiving febuxostat.

Table 3 provides a summary of the primary reason subjects prematurely discontinued participation.

TABLE 3

| Reason for Discontinuation | n | Study |
|---|---|---|
| Withdrew Consent^{a} | 3 | double-blind |
| Adverse Event^{b} | 1 | open-label |
| Noncompliance | 1 | open-label |

^{a}Subjects completed the double-blind study but elected not to enter into the open-label study.
^{b}Preferred Term: Increased Creatinine (Baseline: 1.6 mg/dL, Withdrawal: 2.1 mg/dL, Follow-up Day 163, two weeks off study medication, 1.9 mg/dL)

Table 4 provides a summary of the most frequent adverse events occurring during the study.

TABLE 4
^{a}

| | All Subjects |
|---|---|
| | N = 18 |
| Total Subjects with ≧1 AE | 17 |

MedDRA High Level Term

| | |
|---|---|
| Upper Respiratory Infections | 12 |
| Diarrhea (excluding infectious) | 7 |
| Joint Related Signs and Symptoms | 6 |
| Lower Respiratory Tract and Lung Infections | 5 |
| Musculoskeletal and Connective Tissue | 5 |
| Signs and Symptoms NEC | |
| Non-Site Specific Injuries | 4 |
| Gastrointestinal and Abdominal Pains | 3 |
| (excluding oral and throat) | |
| Edema NEC | 3 |
| Rashes, Eruptions and Exanthems NEC | 3 |
| Urinary Tract Infections | 3 |

NEC = not elsewhere classified
^{a}Adverse events as reported by ≧3 subjects in the open-label study.

Example 1 illustrates that renal function was maintained at generally stable levels in the subjects receiving febuxostat throughout the study.

### EXAMPLE 2

Mice of the species/strain B6C3F1 of an initial age of 6 weeks were dosed via oral gavage with febuxostat suspended in 0.5% methyl cellulose. The daily dose administered was either 0 mg (i.e., the control group), 3 mg, 12 mg, 24 mg, or 48 mg. Histopathological examination of the kidney was carried out after 13-weeks of dosing for vacuolar degeneration of renal proximal tubules (a known naturally occurring change in rodents). The results are shown in Table 5.

TABLE 5

| | Daily Dose | | | | | | | | | |
|---|---|---|---|---|---|---|---|---|---|---|
| | 0 | | | | | | | | | |
| | (Control) | | 3 | | 12 | | 24 | | 48 | |
| | M | F | M | F | M | F | M | F | M | F |
| No. of animals examined | 12 | 12 | 12 | 12 | 12 | 12 | 12 | 12 | 12 | 12 |
| Vacuolar Degeneration | 12 | 3 | 7* | 1 | 5** | 1 | 2** | 0 | 1** | 2 |
| of Renal Proximal | | | | | | | | | | |
| Tubules | | | | | | | | | | |

M = Male F = Female
*p ≦ 0.05 (Dunnett's non-parametric multiple comparison test)
**p ≦ 0.01 (Dunnett's non-parametric multiple comparison test)

Example 2 illustrates that Administration of febuxostat reduced the amount of vacuolar degeneration of the renal proximal tubules in a statistically significant fashion in the male animals studied.

### EXAMPLE 3

Male Wistar rats (295-340 g) were used to produce rats with remnant kidney (RK) as follows. Under light anesthesia with ether, a ⅚ nephrectomy was performed by removal of the right kidney and by selective ligation of 2-3 branches of the left renal artery. Rats were then assigned to one of four treatment groups: Group 1, RK control rats (n=7); Group 2, RK+febuxostat (Fx) rats (n=8); Group 3, RK+oxonic acid (OA) rats (n=6); and Group 4, RK+OA+Fx (n=10). Oxonic acid (OA) (Sigma-Aldrich, St Louis Mo., USA), administered at 750 mg/kg body weight daily by oral gavage, was given starting the day after the ⅚ nephrectomy. Beginning immediately following the surgery, febuxostat was administered in drinking water at 30 mg/L (3-4 mg/kg/day), whereas the respective controls received only drinking water (with 3.5 mg/L of NaCl added to keep an equivalent salt concentration to the Fx-containing water).

All groups were treated for four weeks. Body weight (beginning just before surgery) and food and water intakes were measured daily. Systolic blood pressure, measured in conscious rats by a tail cuff sphygmomanometer, and plasma uric acid (UA) levels were measured at just before surgery (namely, at baseline) and at the end of the four weeks. Proteinuria was measured at baseline and at the end of two and four weeks. A renal micropuncture procedure along with systemic blood pressure monitoring under pentobarbital anesthesia was performed at the end of four weeks followed by morphologic evaluation of the renal preglomerular microvasculature.

### Micropuncture Procedure to Assess Glomerular Hemodynamics

Animals were anesthetized with pentobarbital sodium (30 mg/kg, intraperitoneal (ip)) and placed on a thermoregulated table to maintain body temperature at 37° C. Trachea, jugular veins, femoral arteries and the left ureter were catheterized with polyethylene tubing (PE-240, PE-50, and PE-10). The left kidney was exposed, placed in a Lucite holder, sealed with agar, and covered with Ringer's solution. Mean arterial pressure (MAP) was monitored with a pressure transducer (Model p23 db; Gould, San Juan, Puerto Rico) connected to the catheter in the femoral artery and recorded on a polygraph (Grass Instruments, Quincy, Mass., USA). Blood samples were taken periodically and replaced with blood from a donor rat. Rats were maintained under euvolemic conditions by infusion of 10 mL/kg of body weight of isotonic rat plasma during surgery, followed by an infusion of 25% polyfructosan, at 2.2 ml/h (Inutest; Fresenius Kabi, Linz, Austria). After 60 minutes, five to seven samples of proximal tubular fluid were obtained to determine flow rate and polyfructosan concentrations. Intratubular pressure under free-flow (FF) and stop-flow (SFP) conditions and peritubular capillary pressure (Pc) were measured in other proximal tubules with a servo-null device (Servo Nulling Pressure System; Instrumentation for Physiology and Medicine, San Diego, Calif., USA). Glomerular colloid osmotic pressure was estimated from protein concentrations obtained from blood of the femoral artery (Ca) and surface efferent arterioles (Ce). Polyfructosan was measured in plasma and urine samples by the anthrone-based technique described by Davidson and Sackner in “Simplification of the anthrone method for the determination of inulin in clearance studies,” J Lab Clin Med. 62:351-356 (1963), the contents of which are herein incorporated by reference. In brief, plasma samples were deproteinated first with trichloroacetic acid. After centrifugation, the supernatant was used for polyfructosan measurement. Polyfructosan concentrations in plasma and urine samples were assessed by the addition of anthrone reagent followed by incubation at 45° C. for 50 minutes and reading in a spectrophotometer set at wavelength of 620 nm. Concentrations were calculated by interpolating the absorbance values using a standard curve (0.01-0.05 mg/mL). Total GFR was calculated using the following formula: GFR=(U×V)/P, where U is the polyfructosan concentration in urine, V is urine flow rate, and P is the polyfructosan concentration in plasma.

The volume of fluid collected from individual proximal tubules was estimated from the length of the fluid column in a constant-bore capillary tube of known internal diameter. The concentration of tubular polyfructosan was measured by the microfluorometric method described by Vurek and Pegram in “Fluorometric method for the determination of nanogram quantities of inulin,” Anal Biochem 16:409-419 (1966), the contents of which are herein incorporated by reference. Specifically, using a 8-nL pipette, tubular fluid samples were transferred into capillary cuvettes sealed at one end and containing 3 μL of dimedone reagent (100 mg dimedone in 10 mL of 85% ortho-phosphoric acid). Each cuvette was sealed immediately after adding the samples. Cuvettes were centrifuged five times at maximum speed for five minutes in a hematocrit centrifuge and heated in a boiling water bath for 10 minutes. Fluorescence was measured using a luminescence spectrometer (Series 2; Aminco-Bowman, Rochester N.Y., USA) at excitation and emission wavelengths of 355 and 400 nm, respectively, against the reagent blank as 0% and 10 mg/mL polyfructosan as 100%. For each cuvette, the fluorescence was calculated as the mean of four readings and the holder was rotated arbitrarily between the readings. Polyfructosan concentration was calculated by interpolating the fluorescence values using a standard curve (0.5-2.5 mg/mL). Single-nephron glomerular filtration rate (SNGFR) was calculated using the formula: SNGFR=(TF/P)_{PF}×V, where PF is the concentration of polyfructosan in tubular fluid (TF) and plasma (P), and V is the tubular flow rate which is obtained by timing the collection of tubular fluid (See, Baylis C, et al., “Effects of some vasodilator drugs on transcapillary fluid exchange in renal cortex,” Am J Physiol 230:1148-1158 (1976), the contents of which are herein incorporated by reference).

Protein concentration in afferent and efferent samples was determined according to the method described by Viets et al. in “Determination of serum protein concentration in nanoliter blood samples using fluorescamine or o-phthalaldehyde”, Anal Biochem 88:513-521 (1978), the contents of which are herein incorporated by reference. Specifically, 5 nL of serum was mixed with 5 μL of borate buffer solution containing Brij and mercaptoethanol in a 100-μL glass capillary tube. Additionally, 5 μL of o-phthalaldehyde (OPT) reagent was added. The contents were mixed by centrifuging the capillary tube several times in a hematocrit centrifuge. Fluorescence was measured 30-60 minutes after centrifugation at excitation and emission wavelengths of 362 and 419 nm, respectively, in a luminescence spectrometer (same as described previously). Protein concentration was calculated by interpolating the values of fluorescence obtained in the samples against a standard curve (0.2-1.0 mg/mL). MAP, GFR, glomerular capillary hydrostatic pressure (PGC), single-nephron plasma flow (QA), afferent (AR), efferent (ER) and total (TR) resistances and Kf were calculated with the following equations previously reported in Brenner B M, “Nephron adaptation to renal injury or ablation”, Am J Physiol 249:F324-F337, (1985), the contents of which are herein incorporated by reference:

PGC=SFP+πa, where πa is the colloid osmotic pressure of plasma obtained from femoral artery blood;

QA=SNGFR/SNFF, where SNFF is the single-nephron filtration fraction SNFF=1−(Ca/Ce);

AR=(MAP−PGC/GBF)×(7.962×10¹⁰), where GBF is glomerular blood flow;
GBF=QA/(1−Hct), where Hct is hematocrit;

ER=(PGC−Pc/GBF−SNGFR)×(7.962×10¹⁰);

TR=AR+ER;

Kf=SNGFR/EFP, where EFP is effective filtration pressure; and,

EFP=[(PGC−πa−FF)+(PGC−πe−FF)]/2, where πe is plasma colloid osmotic pressure of blood obtained from surface efferent arterioles.

### Evaluation

Food and water intake were determined daily. Systolic blood pressure (SBP) was measured by a tail-cuff sphygmomanometer using an automated system (XBP-100; Kent Scientific Co, Torrington, Conn., USA) in conscious animals. All animals were preconditioned for blood pressure measurements one week before each experiment. Plasma uric acid was quantified using a commercial kit (Diagnostic Chemicals Ltd, Charlottetown, PEI, Canada). Proteinuria was determined by turbidimetry by the method of trichloroacetic acid as described in Henry R J et al., “Turbidimetric determination of proteins with sulfosalicylic and tricholoroacetic acids”, Proc Soc Exp Biol Med 92:748-751 (1956), the contents of which are herein incorporated by reference.

### Renal Histology and Quantification of Morphology

After the micropuncture study, kidneys were washed by perfusion with phosphate-buffered saline and then fixed with 4% paraformaldehyde. Renal biopsies were embedded in paraffin. Sections of 4-μm thick fixed tissue were stained with periodic acid Schiff (PAS) reagent and Masson's trichrome staining. Arteriolar morphology was assessed by indirect peroxidase immunostaining for alpha-smooth muscle actin (DAKO Corp, Carpinteria, Calif., USA). Renal sections incubated with normal rabbit serum were used as negative controls for immunostaining against alpha smooth-muscle actin.

For each arteriole, the outline of the vessel and its internal lumen (excluding the endothelium) were generated using computer analysis to calculate the total medial area (outline-inline), in 10 arterioles per biopsy. The media/lumen ratio was calculated by the outline/inline relationship (See, Sanchez-Lozada L G et al., “Mild hyperuricemia induces glomerular hypertension in normal rats”, Am J Physiol Renal Physiol 283:F1105-F1110 (2002); Sanchez-Lozada L G, et al., “Mild hyperuricemia induces vasoconstriction and maintains glomerular hypertension in normal and remnant kidney rats,” Kidney Int 67:237-247 (2005), the contents of each are herein incorporated by reference). Quantifications were performed blinded.

The degree of tubulointerstitial fibrosis was quantified in 10 non-crossed fields of cortex (100×) per biopsy. Slides were analyzed by light microscopy (Olympus BX51; Olympus American, Melville, N.Y., USA) and captured by a digital video camera (CoolSnap Pro; Media Cybernetics, Silver Spring, Md., USA). Pictures were processed on a computer and analyzed using Image Pro-Plus (version 5.0; Media Cybernetics, Silver Spring, Md., USA). Taking advantage of the capabilities of color recognition with this software, positive blue-stained areas (fibrosis) were selected and quantified in pixel units; glomeruli and vessels were previously excluded from the field. For each biopsy, the mean amount of positive blue-stained area was calculated by averaging the values from ten examined fields.

### Statistical Analysis

Values are expressed as mean ±standard error of the mean (SEM). Values from the respective four treatment groups were analyzed by one-way analysis of variance (ANOVA). When a p value determined by ANOVA was <0.05, the following comparisons were made using the Bonferroni multiple comparisons test: RK control vs RK+Fx, RK control vs RK+OA, RK control vs RK+OA+Fx and RK+OA vs RK+OA+Fx. The relationship between variables was assessed by correlation analysis.

### Results

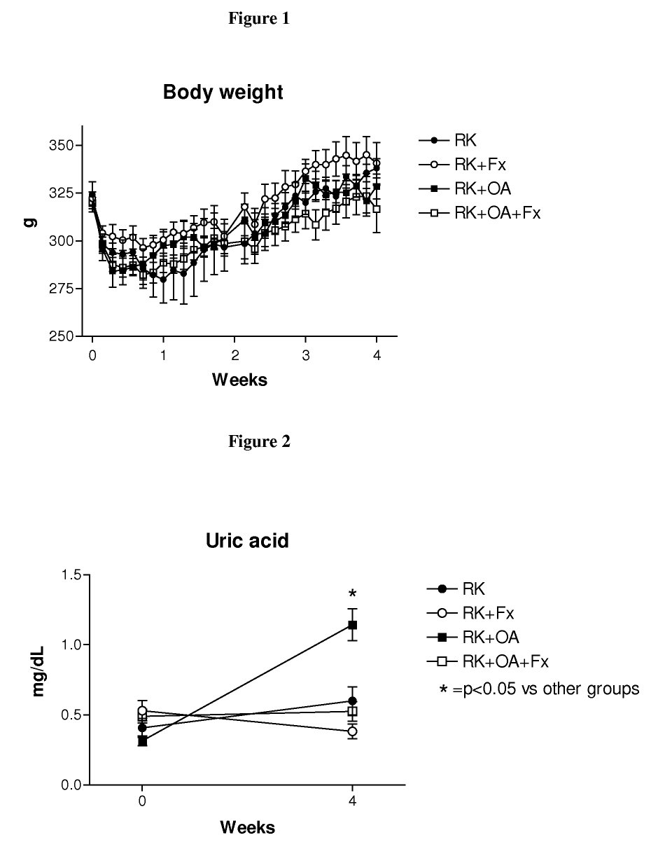

### Body Weight, Food and Water Intake (FIG. 1 and Table 6).

Baseline body weight was similar among all four treatment groups. After surgery, body weight decreased in all treatment groups; this was likely due to reduced food consumption during the first week following the ⅚ nephrectomy. From Week 2 to Week 4, animals ate normally and started to gain body weight. At the end of the study, there were no significant differences in body weight or body weight gain between the four treatment groups. In the two groups treated with febuxostat, rats generally tended to eat slightly less and water intake was generally significantly reduced compared to the RK control or RK+OA groups. Data obtained previously in this specific laboratory (Table 8) and data reported by others (see, Kretschmer B D, et al., “Modulatory role of food, feeding regime and physical exercise on body weight and insulin resistance,” Life Sci 76:1553-1573, (2005)) show that daily water intake in normal male Wistar rats (body weight ≧300 g) is typically 35-40 mL. Based on this information, it is clear from this study that daily water intake increased significantly in RK rats and that water intake was reduced to near normal levels during febuxostat treatment. We do not have a definitive explanation for this behavior, but taste aversion to the drug is a very unlikely possibility, since previously febuxostat exhibited no effect on water intake in normal Sprague-Dawley rats in this specific laboratory. However, it is well known that urinary concentration decreases in response to a reduction of functioning renal mass (see, Hayslett J P, “Functional adaptation to reduction in renal mass,” Physiol Rev 59:137-164 (1979)), and this effect induces polyuria and increased water consumption. In this regard, it has been proposed that the disruption of medullary architecture due to interstitial fibrosis may contribute to the defect in urinary concentration by preventing the generation of a hypertonic medullary interstitium (see, Gilbert R M, et al., “A study of the intrarenal recycling of urea in the rat with chronic experimental pyelonephritis,” J Clin Invest 58:1348-1357 (1976)). Because febuxostat treatment significantly reduced tubulointerstitial fibrosis in RK rats (see below), it is possible that this effect may have had a salutary effect on the urine concentrating ability of the remnant kidney, resulting in normalized water consumption in febuxostat-treated animals.

### Plasma Uric Acid (FIG. 2).

Baseline values of plasma uric acid concentration were similar among all four treatment groups. At the end of four weeks, uric acid in RK rats receiving febuxostat decreased to approximately 63% of the value measured in the RK control rats, but this difference was not statistically significant. As expected, by the end of four weeks plasma uric acid in the RK+OA rats increased significantly by over two-fold relative to the RK control rats. The addition of febuxostat to OA-treated rats prevented the rise of uric acid levels (See, FIG. 2).

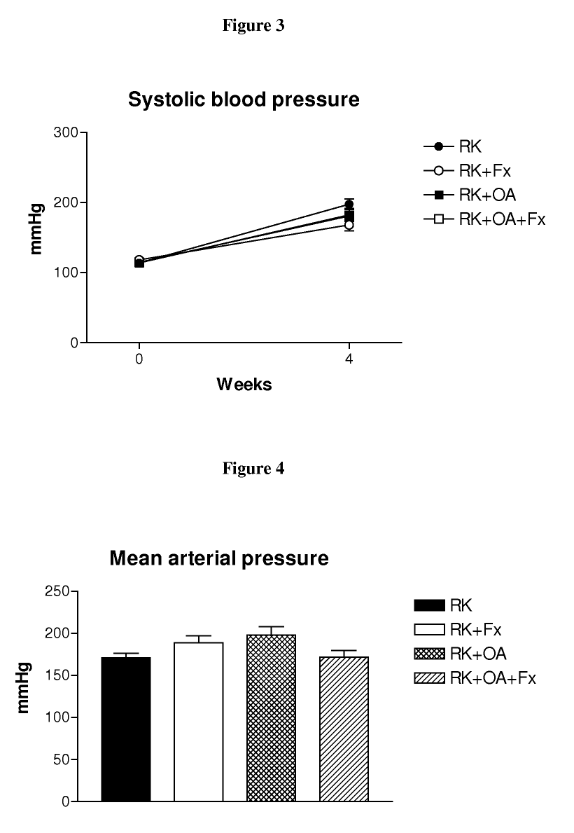

### Blood Pressure (FIGS. 3 and 4).

Values of systolic blood pressure measured by the tail cuff method in conscious animals are summarized in FIG. 3. All treatment groups had similar values at baseline. After four weeks, rats from all four groups developed systemic hypertension to approximately the same degree. This finding was corroborated at the end of the study by the evaluation of mean arterial blood pressure by direct intra-arterial cannulation under anesthesia (See, FIG. 4).

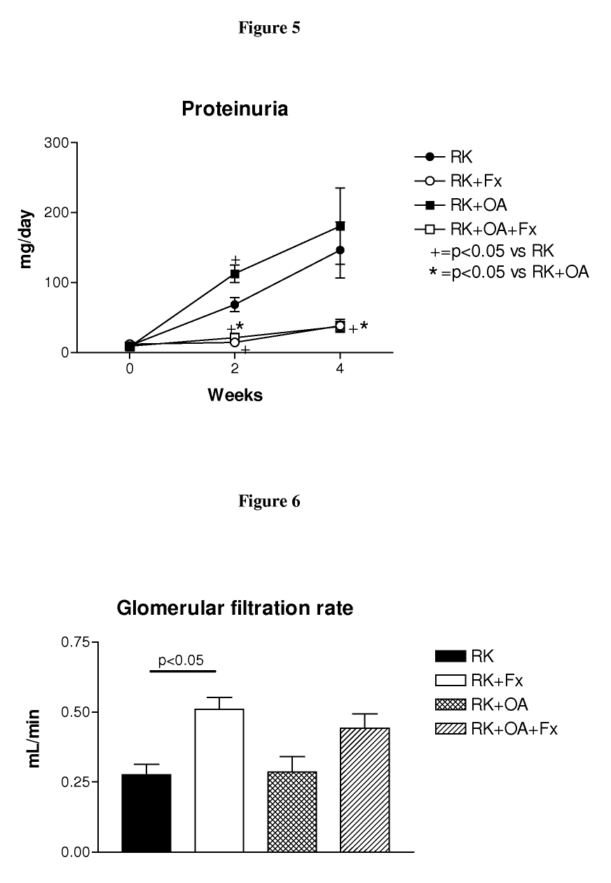

### Proteinuria (FIG. 5).

Values of urinary protein excretion before surgery were similar among the four treatment groups. RK control and RK+OA rats developed a significant proteinuria by Week 2 that continued to increase through Week 4. RK rats with hyperuricemia had, in general, higher proteinuria than the RK rats without hyperuricemia. Treatment with febuxostat prevented the rise of urinary protein excretion in RK rats with and without hyperuricemia. At Week 2, RK+Fx and RK+OA+Fx rats had urinary protein excretion similar to values seen at baseline; and at the end of Week 4, urinary protein excretion was 75-80% lower than the values seen in their respective control groups (See, FIG. 5).

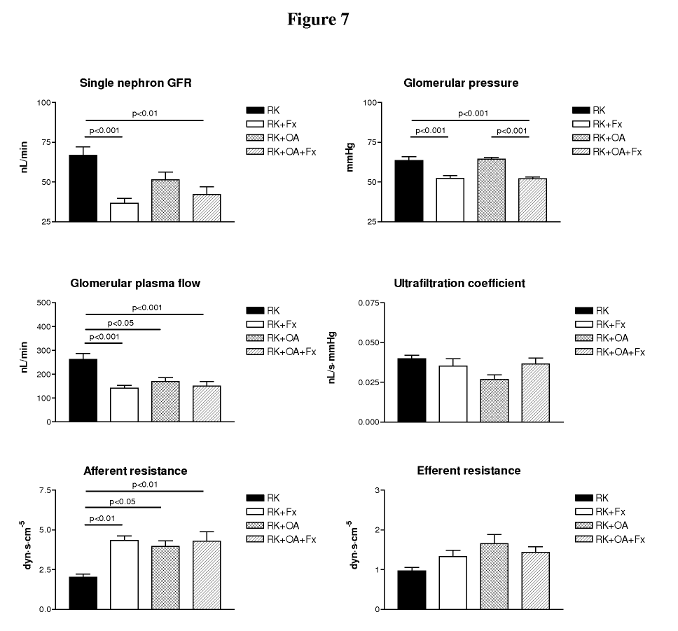

### Glomerular Hemodynamics (FIGS. 6 and 7; Tables 7 and 8)

At the end of the four weeks, glomerular hemodynamics was determined by the micropuncture technique in all animals. As has been previously described in this model of renal damage, subtotal renal ablation induced functional adaptations in remnant nephrons (See, Sanchez-Lozada L G, et al., “Mild hyperuricemia induces vasoconstriction and maintains glomerular hypertension in normal and remnant kidney rats,” Kidney Int 67:237-247 (2005)). Although glomerular filtration rate (GFR) in the RK control rats (0.28±0.04 mL/min; FIG. 6) was markedly reduced, single-nephron GFR (66.8±5.2 mL/min; FIG. 7) increased nearly two-fold compared to historic values obtained in this specific laboratory in a group of normal Wistar rats (See Table 8). Hyperfiltration in remnant nephrons resulted from a significant increase of glomerular pressure and glomerular plasma flow; both of these effects were likely induced by a lack of response of the afferent arterioles to the systemic hypertension, and thus afferent resistance remained low in the face of increased systemic arterial pressure (See, FIG. 7, Tables 7 and 8).

As shown previously in Sprague-Dawley rats (See, Sanchez-Lozada L G, et al., “Mild hyperuricemia induces vasoconstriction and maintains glomerular hypertension in normal and remnant kidney rats,” Kidney Int 67:237-247 (2005)), the presence of hyperuricemia added to the RK model produces additional glomerular hemodynamic changes in Wistar rats. GFR in RK+OA rats was similarly low as in the RK control group (See, FIG. 6); however, single-nephron GFR was lower compared to the RK control group. Moreover, afferent resistance was significantly elevated in the RK+OA rats compared to RK control rats (See, FIG. 7). This cortical vasoconstriction in the RK+OA group was manifested as a significant decrease of glomerular plasma flow despite little or no change in glomerular pressure.

Febuxostat treatment in RK+Fx and RK+OA+Fx rats served to increase GFR compared to the two untreated groups (See, FIG. 6), and it prevented single-nephron hyperfiltration by maintaining normal values of glomerular pressure and glomerular plasma flow. The RK+OA+Fx rats also exhibited higher afferent arteriolar resistances compared to their respective untreated cohorts, suggesting a preserved autoregulatory mechanism in these animals (See, FIG. 7). Consistent with this mechanism is the observation that a negative correlation exists between afferent arteriolar resistance and glomerular pressure (r=−0.57, p<0.001).

At Week 4, positive correlations existed between uric acid and glomerular pressure (r=0.47, p=0.008) and between glomerular pressure and proteinuria (r=0.55, p=0.001).

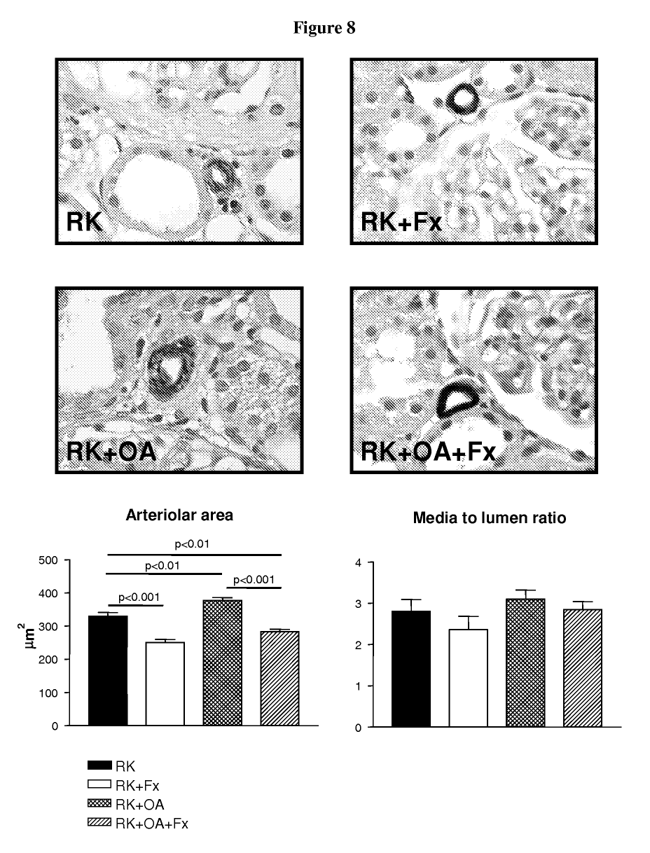

### Renal Arteriolar Morphology (FIG. 8).

Administration of febuxostat to RK animals prevented the thickening of preglomerular vessels observed in the RK control group (See, FIG. 8). RK+OA rats developed additional thickening of the afferent arteriole compared to RK control animals; this alteration was prevented by febuxostat treatment (See, FIG. 8). Furthermore, the following positive correlations were found to exist: uric acid vs arteriolar area (r=0.69, p<0.0001) and arteriolar area vs glomerular pressure (r=0.66, p<0.0001). There were no statistically significant differences in the media/lumen (M/L) ratios among the various groups (See, FIG. 8); however, there was a tendency for the M/L ratio to be lower in febuxostat-treated rats compared to their respective untreated cohorts.

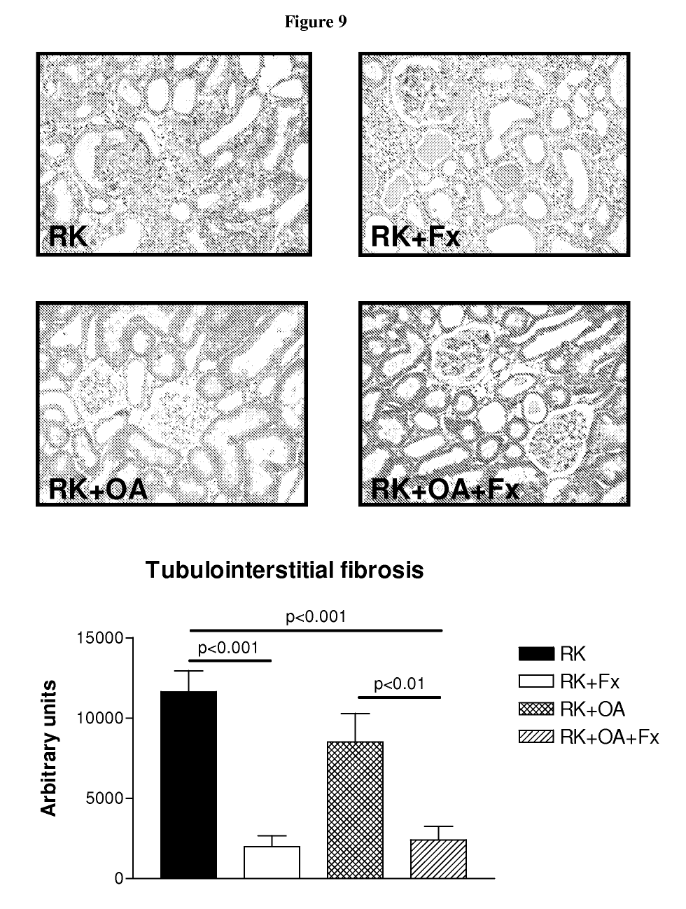

### Tubulointerstitial Fibrosis (FIG. 9).

The RK control and RK+OA groups developed a similar degree of tubulointerstitial (TI) fibrosis. Treatment with febuxostat significantly decreased this structural alteration in both RK and RK+OA rats. Additionally, the following positive correlations were identified: uric acid vs TI fibrosis (r=0.44, p=0.02); TI fibrosis vs proteinuria (r=0.74, p<0.0001); glomerular pressure vs TI fibrosis (r=0.65, p=0.0001); and TI fibrosis vs arteriolar area (r=0.67, p<0.0001).

Table 6 provides a summary of the effect of febuxostat on body weight, food and water intake in remnant kidney rats with and without coexisting hyperuricemia

| | | RK | | | RK + OA + |
|---|---|---|---|---|---|
| | | control | RK + Fx | RK + OA | Fx |
| Parameter | Time | (n = 7) | (n = 8) | (n = 6) | (n = 10) |
| BW (g) | Baseline | 324.3 ± 1.1 | 322.3 ± 3.4 | 323.0 ± 7.9 | 319.7 ± 2.9 |
| | End of Week 4 | 338.0 ± 5.0 | 340.6 ± 10.8 | 328.5 ± 6.6 | 316.7 ± 12.3 |
| BW Gain | End of Week 4 | 13.7 ± 5.0 | 18.4 ± 8.2 | 5.5 ± 10.3 | −3.0 ± 11.5 |
| (from baseline) (g) | | | | | |
| Daily Food | Week 1 | 11.5 ± 1.7 | 8.6 ± 1.5 | 14.2 ± 2.1 | 8.7 ± 1.7 |
| Intake (g)¹ | Week 2 | 17.4 ± 0.9 | 15.4 ± 0.5 | 19.7 ± 0.7 | 16.0 ± 0.7# |
| | Week 3 | 19.3 ± 0.8 | 20.2 ± 0.5 | 21.3 ± 1.0 | 18.4 ± 0.5 |
| | Week 4 | 22.4 ± 0.7 | 22.4 ± 1.3 | 20.7 ± 0.9 | 18.6 ± 0.5* |
| Daily Water | Week 1 | 38.0 ± 2.4 | 31.5 ± 1.4 | 44.3 ± 3.5 | 30.2 ± 2.8# |
| Intake (mL)¹ | Week 2 | 50.5 ± 3.0 | 32.6 ± 1.2* | 58.6 ± 1.4 | 40.6 ± 2.6*# |
| | Week 3 | 52.4 ± 1.2 | 37.2 ± 2.5* | 57.2 ± 2.6 | 38.6 ± 1.0*# |
| | Week 4 | 55.5 ± 2.3 | 39.4 ± 1.6* | 48.6 ± 3.0 | 40.5 ± 1.6* |

RK = remnant kidney; Fx = febuxostat; OA = oxonic acid (used to induce hyperuricemia).
¹Mean ± SEM was calculated from the average of daily food or water intake over one week for each animal.
*indicates significant difference from RK control group.
#indicates significant difference from RK + OA group.

Table 7 describes the effect of febuxostat on glomerular hemodynamics in remnant kidney rats with and without coexisting hyperuricemia

| | Treatment Group^{a} | | | |
|---|---|---|---|---|
| | | | | RK + OA + |
| | RK control | RK + Fx | RK + OA | Fx |
| Parameter | (n = 7) | (n = 8) | (n = 6) | (n = 10) |
| MAP (mmHg) | 171 ± 5 | 189 ± 8 | 198 ± 10 | 172 ± 8 |
| PGC (mmHg) | 63.6 ± 2.3 | 52.2 ± 1.9* | 64.4 ± 1.1 | 52.0 ± 1.2*^{#} |
| GFR (mL/min) | 0.28 ± 0.04 | 0.51 ± 0.04* | 0.29 ± 0.06 | 0.44 ± 0.05 |
| SNGFR | 66.8 ± 5.2 | 36.7 ± 3.1* | 51.3 ± 4.8 | 42.2 ± 4.9* |
| (nL/min) | | | | |
| QA (nL/min) | 263 ± 25 | 142 ± 11* | 170 ± 16* | 151 ± 19* |
| AR (dyn · s · cm⁻⁵) | 2.02 ± 0.21 | 4.33 ± 0.30* | 3.95 ± 0.36* | 4.30 ± 0.60* |
| ER (dyn · s · cm⁻⁵) | 0.97 ± 0.09 | 1.33 ± 0.16 | 1.66 ± 0.22 | 1.43 ± 0.15 |
| Kf (nL/s · mmHg) | 0.040 ± 0.002 | 0.035 ± 0.005 | 0.027 ± 0.003 | 0.037 ± 0.004 |

RK = remnant kidney; Fx = febuxostat; OA = oxonic acid (used to induce hyperuricemia).
MAP: mean arterial pressure; PGC: glomerular capillary pressure; GFR: glomerular filtration rate; SNGFR: single-nephron GFR; QA: glomerular plasma flow; AR: afferent resistance; ER: efferent resistance; Kf: ultrafiltration coefficient.
*indicates significant difference from RK control group.
^{#}indicates significant difference from RK + OA group.

TABLE 8
Table 8 describes historic control values from normal male wistar rats.
Historic Control Values From Normal Male Wistar Rats

| | |
|---|---|
| Parameter | Sample Group |

| | | |
|---|---|---|
| Sample Size | 6 | 6 |
| Body weight (g) | 353 ± 6 | 317 ± 6 |
| Daily Water Intake (mL) | nd | 39 ± 1 |
| Daily Food Intake (g) | nd | 13 ± 1 |
| Uprot (mg/day) | 16 ± 1.5 | nd |
| SBP (mmHg) | 118 ± 3.4 | nd |
| MAP (mmHg) | 118 ± 2.7 | nd |
| PGC (mmHg) | 50.3 ± 1.2 | nd |
| GFR (in one kidney, | 0.81 ± 0.10 | nd |
| mL/min) | | |
| SNGFR (nL/min) | 34.4 ± 2.8 | nd |
| QA (nL/min) | 112 ± 9.5 | nd |
| AR (dyn · s · cm⁻⁵) | 2.6 ± 0.2 | nd |
| ER (dyn · s · cm⁻⁵) | 1.8 ± 0.2 | nd |
| Kf (nL/s · mmHg) | 0.042 ± 0.006 | nd |

nd = no data

The results of the above study described in this Example 3 demonstrate that febuxostat treatment prevented proteinuria and renal injury in RK rats with and without coexisting hyperuricemia. Moreover, because febuxostat helped preserve preglomerular vessel morphology, normal glomerular pressure was maintained even in the presence of systemic hypertension. This study highlights the importance of preservation of the autoregulatory capacity of remnant nephrons in order to retard the progression of renal disease. Therefore, febuxostat treatment reduces the functional and structural alterations induced by the progressive and extensive loss of renal tissue in a rat model of chronic renal disease alone or in combination with coexisting hyperuricemia.

While the invention has been described by reference to certain presently preferred embodiments, it will be understood that modifications and variations thereof apparent to those skilled in the art are intended to be included within the scope of the invention.

What is claimed is:

1. A method of preserving renal function in a subject in need thereof, the method comprising the step of:
administering to the subject a therapeutically effective amount of at least one compound, wherein said at least one compound is a xanthine oxidoreductase inhibitor or a pharmaceutically acceptable salt thereof.

2. The method of claim 1, wherein the xanthine oxidoreductase inhibitor is selected from the group consisting of: 2-[3-cyano-4-(2-methylpropoxy)phenyl]-4-methylthiazole-5-carboxylic acid, 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid, 1-(3-cyano-4-(2,2-dimethylpropoxy)phenyl)-1H-pyrazole-4-carboxylic acid, 1-3-cyano-4-(2,2-dimethylpropoxy)phenyl]-1H-pyrazole-4-carboxylic acid, pyrazolo[1,5-a]-1,3,5-triazin-4-(1H)-one, 8-[3-methoxy-4-(phenylsulfinyl)phenyl]-sodium salt (±), 3-(2-methyl-4-pyridyl)-5-cyano-4-isobutoxyphenyl)-1,2,4-triazole and a pharmaceutically acceptable salt thereof.

3. The method of claim 1, wherein the subject has hyperuricemia, gout, acute gouty arthritis, chronic gouty joint disease, tophaceous gout, uric acid nephropathy or nephrolithiasis.

4. The method of claim 1, wherein the subject has a progressive renal disease.

5. The method of claim 1, wherein the subject's GFR is maintained at a level of at least approximately 75% or greater when compared to the subject's baseline GFR level.

6. A method of preserving renal function in a subject in need thereof, the method comprising the step of:
administering to the subject a therapeutically effective amount of a compound or a pharmaceutically acceptable salt thereof, wherein said compound comprises the formula: 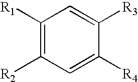
wherein R₁ and R₂ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, a phenylsulfinyl group or a cyano (—CN) group;
wherein R₃ and R₄ are each independently a hydrogen or A, B, C or D as shown below: 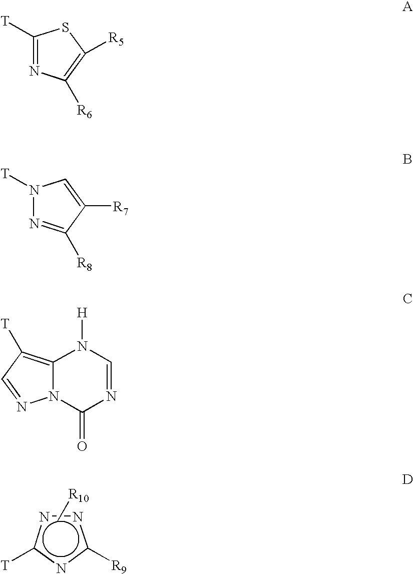
wherein T connects A, B, C or D to the aromatic ring shown above at R₁, R₂, R₃ or R₄.
wherein R₅ and R₆ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;
wherein R₇ and R₈ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;
wherein R₉ is an unsubstituted pyridyl group or a substituted pyridyl group; and
wherein R₁₀ is a hydrogen or a lower alkyl group, a lower alkyl group substituted with a pivaloyloxy group and in each case, R₁₀ bonds to one of the nitrogen atoms in the 1,2,4-triazole ring shown above.

7. The method of claim 6, wherein the compound is 2-[3-cyano-4-(2-methylpropoxy)phenyl]-4-methylthiazole-5-carboxylic acid or a pharmaceutically acceptable salt thereof.

8. The method of claim 6, wherein the compound is 2-[3-cyano-4-(3-hydroxy-2-methylpropoxy)phenyl]-4-methyl-5-thiazolecarboxylic acid or a pharmaceutically acceptable salt thereof.

9. The method of claim 6, wherein the compound is 2-[3-cyano-4-(2-hydroxy-2-methylpropoxy)phenyl]-4-methyl-5-thiazolecarboxylic acid or a pharmaceutically acceptable salt thereof.

10. The method of claim 6, wherein the compound is 2-(3-cyano-4-hydroxyphenyl)-4-methyl-5-thiazolecarboxylic acid or a pharmaceutically acceptable salt thereof.

11. The method of claim 6, wherein the compound is 2-[4-(2-carboxypropoxy)-3-cyanophenyl]-4-methyl-5-thiazolecarboxylic acid or a pharmaceutically acceptable salt thereof.

12. The method of claim 6, wherein the compound is 1-3-cyano-4-(2,2-dimethylpropoxy)phenyl]-1H-pyrazole-4-carboxylic acid or a pharmaceutically acceptable salt thereof.

13. The method of claim 6, wherein the compound is pyrazolo[1,5-a]-1,3,5-triazin-4-(1H)-one, 8-[3-methoxy-4-(phenylsulfinyl)phenyl]-sodium salt (±).

14. The method of claim 6, wherein the compound is 3-(2-methyl-4-pyridyl)-5-cyano-4-isobutoxyphenyl)-1,2,4-triazole or a pharmaceutically acceptable salt thereof.

15. The method of claim 6, wherein the subject has hyperuricemia, gout, acute gouty arthritis, chronic gouty joint disease, tophaceous gout, uric acid nephropathy or nephrolithiasis.

16. The method of claim 6, wherein the subject has a progressive renal disease.

17. The method of claim 6, wherein the subject's GFR is maintained at a level of at least approximately 75% or greater when compared to the subject's baseline GFR level.

18. A method of preserving renal function in a subject in need of thereof, the method comprising the step of:
administering to the subject a therapeutically effective amount of a compound or a pharmaceutically acceptable salt thereof, wherein said compound comprises the formula: 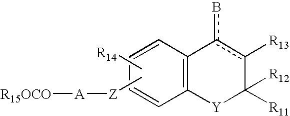
wherein R₁₁ and R₁₂ are each independently a hydrogen, a substituted or unsubstituted lower alkyl group, a substituted or unsubstituted phenyl, or R₁₁ and R₁₂ may together form a four- to eight-membered carbon ring together with the carbon atom to which they are attached;
wherein R₁₃ is a hydrogen or a substituted or unsubstituted lower alkyl group;
wherein R₁₄ is one or two radicals selected from a group consisting of a hydrogen, a halogen, a nitro group, a substituted or unsubstituted lower alkyl, a substituted or unsubstituted phenyl, —OR₁₆ and —SO₂NR₁₇R_{17′}, wherein R₁₆ is a hydrogen, a substituted or unsubstituted lower alkyl, a phenyl-substituted lower alkyl, a carboxymethyl or ester thereof, a hydroxyethyl or ether thereof, or an allyl; R₁₇ and R_{17′} are each independently a hydrogen or a substituted or unsubstituted lower alkyl;
wherein R₁₅ is a hydrogen or a pharmaceutically active ester-forming group;
wherein A is a straight or branched hydrocarbon radical having one to five carbon atoms;
wherein B is a halogen, an oxygen, or a ethylenedithio;
wherein Y is an oxygen, a sulfur, a nitrogen or a substituted nitrogen;
wherein Z is an oxygen, a nitrogen or a substituted nitrogen; and
the dotted line refers to either a single bond, a double bond, or two single bonds.

19. The method of claim 18, wherein the subject has hyperuricemia, gout, acute gouty arthritis, chronic gouty joint disease, tophaceous gout, uric acid nephropathy, or nephrolithiasis.

20. The method of claim 18, wherein the subject has a progressive renal disease.

21. The method of claim 18, wherein the subject's GFR is maintained at a level of at least approximately 75% or greater when compared to the subject's baseline GFR level.
